(19) United States
(12) Patent Application Publication
(10) Pub. No.: US 2024/0417678 A1
(43) Pub. Date: Dec. 19, 2024

(54) METHOD FOR THREE-DIMENSIONAL CELL CULTURE USING CELL GROWTH PROMOTING COLLOID AND MASS PRODUCTION OF HIGH-CONCENTRATION EXTRACELLULAR COMPOSITION USING THE SAME

(71) Applicant: 3D GLOBAL BIOTECH INC., New Taipei City (TW)

(72) Inventors: Keng-Liang Ou, Taipei City (TW); Hsu-An Pan, Yilan County (TW)

(21) Appl. No.: 18/212,339

(22) Filed: Jun. 21, 2023

(30) Foreign Application Priority Data
Jun. 15, 2023 (TW) 112122304

(51) Int. Cl. C12N 5/00

(52) U.S. Cl. CPC C12N 5/0062; C12N 2511/00; C12N 2513/00; C12N 2537/10

(57) ABSTRACT

A method for three-dimensional cell culture using a cell growth promoting colloid and a mass production of high-concentration extracellular composition using the same are provided. The method for three-dimensional cell culture using the cell growth promoting colloid includes: providing a culture colloid to mix with cells to embed the cells therein; performing a crosslinking treatment to the culture colloid embedding the cells, so that the culture colloid that is crosslinked has a three-dimensional structure and the cells are suspended in the culture colloid that is crosslinked; and performing cell culture.

### CROSS-REFERENCE TO RELATED PATENT APPLICATION

This application claims the benefit of priority to Taiwan Patent Application No. 112122304, filed on Jun. 15, 2023. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a method for culturing cells, and more particularly to a method for three-dimensional cell culture using a cell growth promoting colloid and using the same to mass produce a high-concentration extracellular composition.

### BACKGROUND OF THE DISCLOSURE

The exosome is an important component of the extracellular composition with a diameter of about 30 nm to 150 nm released by eukaryotic cells of animals, plants, fungi, etc. The exosome contains carbohydrates, lipids, proteins, mRNA, DNA, etc., and plays an important function in intercellular signal transduction. In addition, the exosome is also associated with cell growth, inflammation, cancer cell metastasis and progression.

However, the secretion, production, and biological activity of the exosome may be affected by factors such as the health of the cells and conventional two-dimensional culture environment, causing difficulties in effectively mass production and reducing batch-to-batch variation.

Therefore, how to improve the survival rate of cells and improve the yield and batch variability of extracellular composition through improvements in cell culture methods to overcome the aforementioned problems has become an issue to be addressed in the relevant field.

### SUMMARY OF THE DISCLOSURE

In response to the above-referenced technical inadequacies, the present disclosure provides a method for three-dimensional cell culture using a cell growth promoting colloid and a mass production of high-concentration extracellular composition using the same.

In order to solve the above-mentioned problems, one of the technical aspects adopted by the present disclosure is to provide a method for three-dimensional cell culture using a cell growth promoting colloid, which includes: providing a culture colloid to mix with cells to embed the cells therein; performing a crosslinking treatment to the culture colloid embedding the cells, so that the culture colloid that is crosslinked has a three-dimensional structure and the cells are suspended in the culture colloid that is crosslinked; and performing cell culture.

In order to solve the above-mentioned problems, another one of the technical aspects adopted by the present disclosure is to provide a method for a mass production of high-concentration extracellular composition using the method for three-dimensional cell culture using the cell growth promoting colloid aforementioned, which includes: collecting a conditioned medium; and performing filtration the condition medium.

In order to solve the above-mentioned problems, yet another one of the technical aspects adopted by the present disclosure is to provide an extracellular composition, which is obtained using the method for the mass production of high-concentration extracellular composition aforementioned.

Therefore, in the method for three-dimensional cell culture using the cell growth promoting colloid and the mass production of high-concentration extracellular composition using the same provided by the present disclosure, by virtue of “performing the crosslinking treatment to the culture colloid embedding the cells, so that the culture colloid that is crosslinked has the three-dimensional structure and the cells are suspended in the culture colloid that is crosslinked,” damage to the cells can be reduced during the three-dimensional cell culture and three-dimensional growth of the cells can be supported in the culture colloid.

Further. the extracellular composition obtained using the method for three-dimensional cell culture using the cell growth promoting colloid of the present disclosure has an effective reduction in particle size and a significant increase in total protein concentration. Accordingly, a yield of the extracellular composition can be effectively improved, and an excellent effect of enhancing cell growth can be achieved.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a flowchart of a method for three-dimensional cell culture using a cell growth promoting colloid according to a first embodiment of the present disclosure;

FIG. 2 is a curve diagram showing rheological curves of the cell growth promoting colloid at different crosslinking times according to the first embodiment of the present disclosure;

FIG. 3 is a histogram showing differences in cell proliferation when cell cultures carried out in different ways according to the first embodiment of the present disclosure;

FIG. 4 is a flowchart of a method for a mass production of high-concentration extracellular composition using the method for three-dimensional cell culture using the cell growth promoting colloid according to a second embodiment of the present disclosure;

FIG. 5 is another flowchart of the method for the mass production of high-concentration extracellular composition using the method for three-dimensional cell culture using the cell growth promoting colloid according to the second embodiment of the present disclosure;

FIG. 6 is a histogram showing differences in total protein concentrations when cell cultures are carried out in different ways according the second embodiment of the present disclosure;

FIG. 7 is a curve diagram showing a particle size distribution of an extracellular composition obtained from a two-dimensional cultured cells according to the second embodiment of the present disclosure;

FIG. 8 is a curve diagram showing a particle size distribution of an extracellular composition obtained from a three-dimensional cultured cells according to the second embodiment of the present disclosure;

FIG. 9 is a histogram showing differences in total protein concentrations of the extracellular compositions when cell cultures are carried out in different ways according the second embodiment of the present disclosure; and

FIG. 10 is a histogram showing differences in cell proliferation affected by the extracellular compositions when cell cultures carried out in different ways according to the second embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

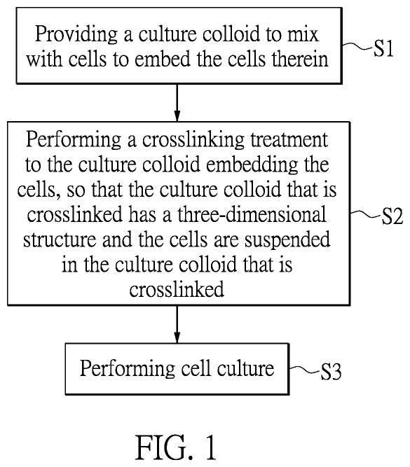

Referring to FIG. 1, a first embodiment of the present disclosure provides a method for three-dimensional cell culture using a cell growth promoting colloid. The method at least includes the following steps.

Step S1: providing a culture colloid to mix with cells to embed the cells therein. In one particular embodiment, the culture colloid can be selected from a group consisting of sodium alginate, gelatin, gelatin methacrylamide, carboxymethyl cellulose, collagen, hyaluronic acid, gum tragacanth, gum arabic, xanthan gum, pectin, guar gum, carrageenans, and any combination thereof, and a concentration of the culture colloid is 0.1% to 10%, but the present disclosure is not limited thereto.

Further, the culture colloid can also include a cell growth promoter to enhance cell proliferation and increase a number of cells. In one particular embodiment, the cell growth promoter can be selected from a group consisting of extracellular matrix (ECM), collagen, fibronectin, laminin, elastin, growth factor, extracellular vesicle, and any combination thereof, and a concentration of the cell growth promoter is 0.1% to 10%, but the present disclosure is not limited thereto.

In one particular embodiment of the present disclosure, the method for three-dimensional cell culture using the cell growth promoting colloid can be used to culture human-derived and animal-derived mesenchymal stem cells (MSCs), fibroblasts, epithelial cells, endothelial cells, epidermal stem cells, dental pulp/gingival stem cells, hair follicle dermal papilla cells, keratinocytes, or melanocytes. It should be noted that, the cell types for which the method for three-dimensional cell culture using the cell growth promoting colloid of the present disclosure is applicable can be adjusted according to practical applications, but the present disclosure in not limited thereto. In addition, a number of cells used in the method for three-dimensional cell culture using the cell growth promoting colloid of the present disclosure can be 1×10⁵ cells/mL to 2×10⁶ cells/mL, but the present disclosure is not limited thereto.

Step S2: performing a crosslinking treatment to the culture colloid embedding the cells, so that the culture colloid that is crosslinked has a three-dimensional structure and the cells are suspended in the culture colloid that is crosslinked. In one particular embodiment, a three-dimensional cell culture can be carried out by three-dimensional flat layered colloid culture. For example, the cells can be mixed with the culture colloid to be embedded therein and then the culture colloid is subjected to the crosslinking treatment, so that the cells are suspended in a flat layered culture colloid that is crosslinked. In one preferred embodiment, the culture colloid embedding the cells is subject to the crosslinking treatment and spread on a surface of a culture dish until a height of the culture colloid is 10 mm to 50 mm, and then 0.1 mL/cm² to 0.5 mL/cm² culture medium is added for three-dimensional cell culture. It should be noted that the present disclosure merely describes the differences from conventional cell culture techniques, other procedures of cell culture not described are conventional techniques in the related field and can be adjusted according to practical applications, and will not be reiterated herein.

In one particular embodiment, the three-dimensional cell culture can be carried out by three-dimensional spherical colloid culture. For example, the cells can be mixed with the culture colloid to be embedded therein and then the culture colloid is subjected to the crosslinking treatment, and a spherical culture colloid with a diameter of 0.1 mm to 1 mm is formed by, for example but not limited to, syringe injection, a dispensing machine, or a three-dimensional (3D) printer, so that the cells are suspended in the spherical culture colloid that is crosslinked.

In one particular embodiment, the three-dimensional cell culture can be carried out by three-dimensional printing colloid culture. For example, the cells can be mixed with the culture colloid to be embedded therein and then the culture colloid is subjected to the crosslinking treatment, and the culture colloid mixed with the cells can be formed into a three-dimensional mesh culture colloid by the 3D printer, so that the cells are suspended in the three-dimensional mesh culture colloid that is crosslinked. It should be noted that, a three-dimensional structure formed by the 3D printer can be adjusted according to practical applications, but the present disclosure is not limited thereto.

In one particular embodiment, the crosslinking treatment is performed by mixing the culture colloid with a crosslinking agent, so that the colloid is changed from an original gel state to a jelly-like solid state to obtain the culture colloid that is crosslinked. In the present disclosure, the crosslinking agent can be calcium chloride, barium chloride, zinc chloride, calcium carbonate, calcium sulfate, or calcium lactate. In one preferred embodiment, the crosslinking agent is 5 vol % to 25 vol % of a calcium chloride solution, but the present disclosure is not limited thereto.

Step S3: performing cell culture. Further, the three-dimensional cell culture is performed by culturing the cells in the culture medium. In one particular embodiment, the culture medium can be DMEM-HG, DMEM-F12, DMEM, MEM alpha, DMEM-LG, etc., which can be adjusted according to practical applications, but the present disclosure is not limited thereto. In addition, conditions of cell culture, such as temperature and CO2 concentration, can be adjusted according to practical applications, but the present disclosure is not limited thereto.

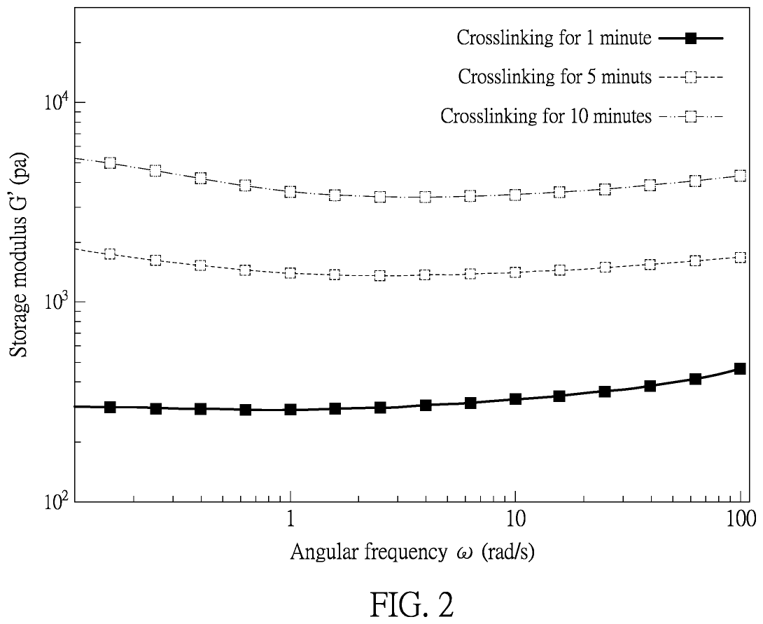

In one particular embodiment, rheological properties of the culture colloid that is crosslinked is further analyzed by dynamic frequency scanning with a rheometer to measure storage modulus (G′). As shown in FIG. 2, the measured storage modulus (G′) value increases with increasing crosslinking time. In one preferred embodiment, the storage modulus of the culture colloid is 1000 Pa to 5000 Pa. If it is higher than such range, it is not conductive to cell growth. If it is lower than such range, the culture colloid cannot effectively embed the cells, such that it is easy for the cells to penetrate the culture colloid.

As a comparative example, the difference from the method for three-dimensional cell culture using the cell growth promoting colloid of the present disclosure is that, the cells are seeded in the culture dish at 1×10⁵ cells/mL to 2×10⁶ cells/mL, so that the cells are adhered to the culture dish for two-dimensional culture. It should be noted that, the culture medium and the culture conditions of the comparative example are the same as those of the three-dimensional cell culture of the present disclosure, and will not be reiterated herein.

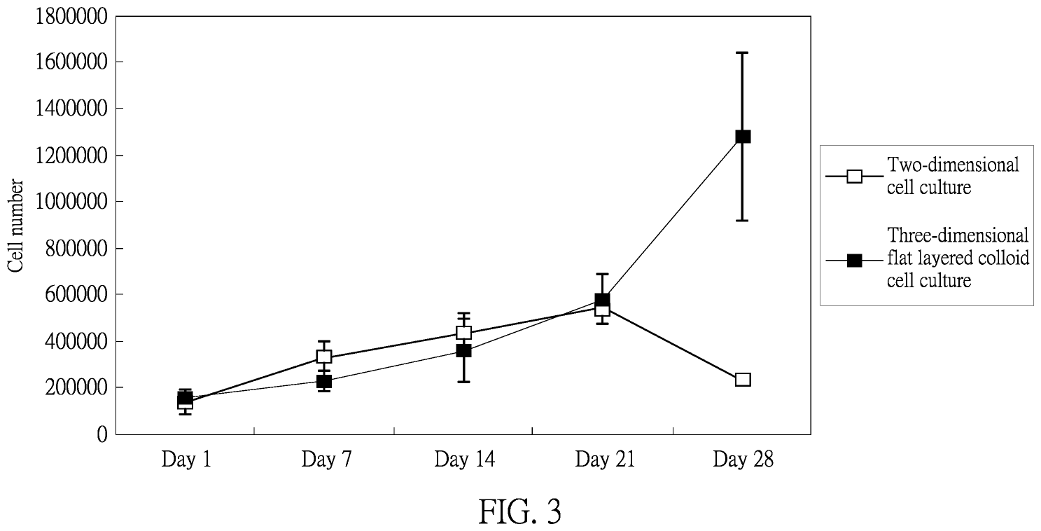

As shown in FIG. 3, when the three-dimensional cell culture is carried out, cell proliferation increased gradually with time. Especially, the cell proliferation significantly increased by more than two times between day 21 and day 28 of culture. On the other hand, when the two-dimensional culture is carried out, a degree of cell proliferation is comparable to that of the three-dimensional cell culture up to day 21, but is significantly decreased by more than half between day 21 and day 28 of culture. That is, compared with the conventional two-dimensional cell culture method, the method for three-dimensional cell culture using the cell growth promoting colloid of the present disclosure has a beneficial effect of significantly increasing the number of cells.

However, the aforementioned details are disclosed for exemplary purposes only, and are not meant to limit the scope of the present disclosure.

### Second Embodiment

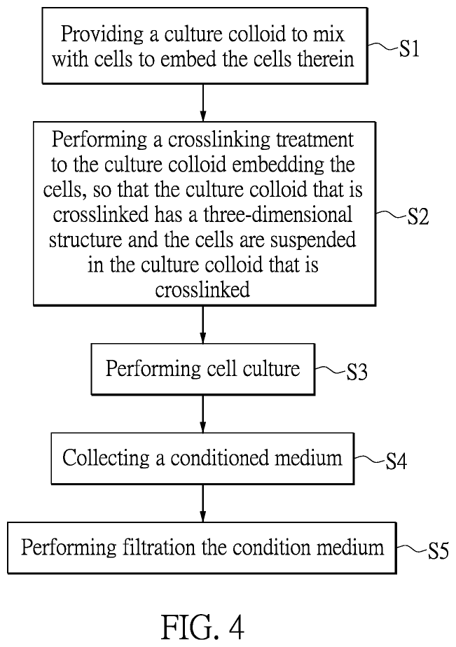

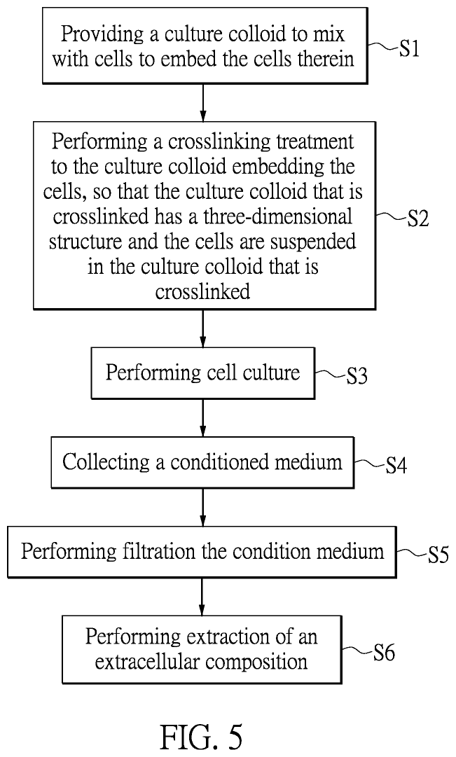

Referring to FIG. 4 and FIG. 5, a second embodiment of the present disclosure provides a method for a mass production of extracellular composition using the method for three-dimensional cell culture using the cell growth promoting colloid, which at least includes, in addition to steps S1 to S3 in the aforementioned first embodiment, the following steps.

Step S4: collecting a conditioned medium, i.e., the culture medium in which the cells were cultured. In one particular embodiment, after the cells are cultured for at least seven to fourteen days, the culture medium is removed and the cells are washed two times to three times with PBS, and the cells are further cultured with a culture medium without nutrients (e.g., fetal bovine serum (FBS), culture supplement, etc). Afterwards, the conditioned medium is collected every one to three days, and a fresh culture medium without nutrients is then added. Such the step is repeated three to five times to pool the conditioned medium. In should be noted that, the cell culture time and the number of times of collecting the conditioned medium can be adjusted according to practical applications, but the present disclosure is not limited thereto. In addition, the collected conditioned medium can be used directly, but the present disclosure is not limited thereto.

Step S5: performing filtration the condition medium. In one particular embodiment, the collected conditioned medium can be further mixed with 500 mM to 1000 mM crosslinking reagent and react for eight hours to sixteen hours at 4° C. Then, centrifugation is conducted at 1000 g for 20 minutes to 30 minutes, and a 0.22 μm filter is used to filter cell debris and the colloid to obtain the conditioned medium rich in an extracellular composition. In addition, as shown in FIG. 4, the conditioned medium rich in the extracellular composition can be used directly before proceeding to step S6 described below, but the present disclosure is not limited thereto.

Step S6: performing extraction of the extracellular composition. In one particular embodiment, the conditioned medium rich in the extracellular composition obtained after step S5 can be further concentrated by 10 times to 50 times through a hollow fiber membrane (e.g., polyethersulfone filter material with a molecular weight cut-off (MWCO) of 1 kDa to 500 kDa) of a tangential flow filtration system to obtain a high-concentration extracellular composition. It should be noted that, the extraction of the extracellular composition can also be adjusted according to practical applications. For example, the high-concentration extracellular composition can be obtained by ultra-high speed centrifugation, polymer precipitation, size exclusion chromatography, antigen-antibody specificity isolation, etc., but the present disclosure is not limited thereto.

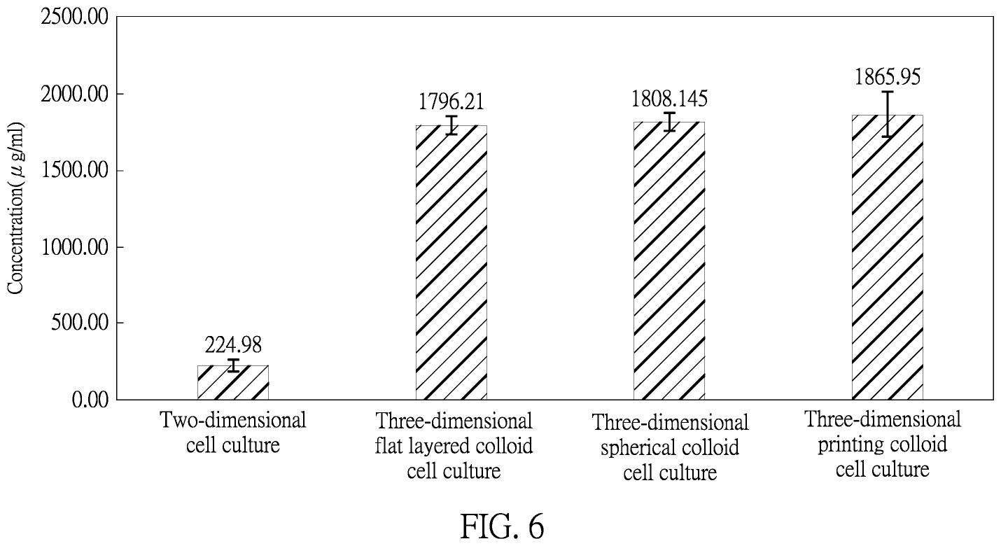

Similarly, as a comparative example, except for the step of the two-dimensional culture, other steps are the same as the method for the mass production of extracellular composition using the method for three-dimensional cell culture using the cell growth promoting colloid of the present disclosure. A total protein concentration is measured with Bio-Rad protein assay kit. As shown in FIG. 6, a total protein of the three-dimensional cultured cells (including the three-dimensional flat layered colloid cell culture, the three-dimensional spherical colloid cell culture, and the three-dimensional printing colloid cell culture as shown in FIG. 6) is much higher than a total protein concentration of the two-dimensional cell culture, indicating that the method for the mass production of extracellular composition of the present disclosure has a higher yield compared with the conventional method.

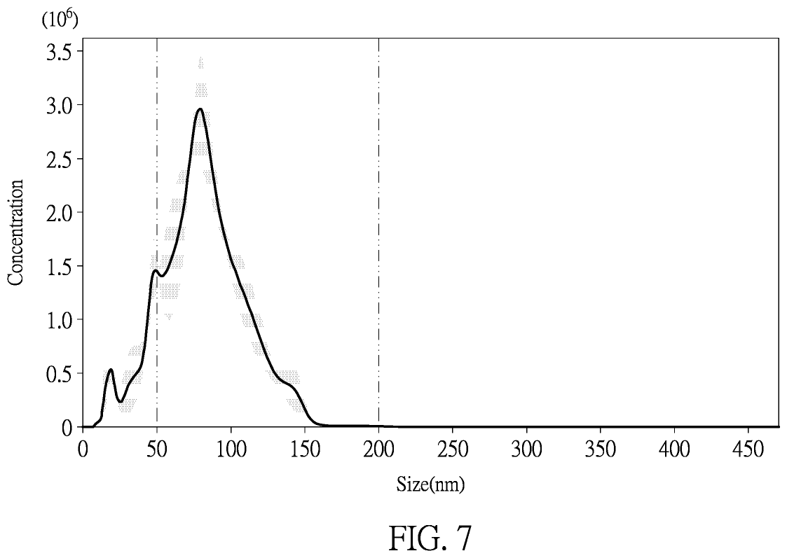

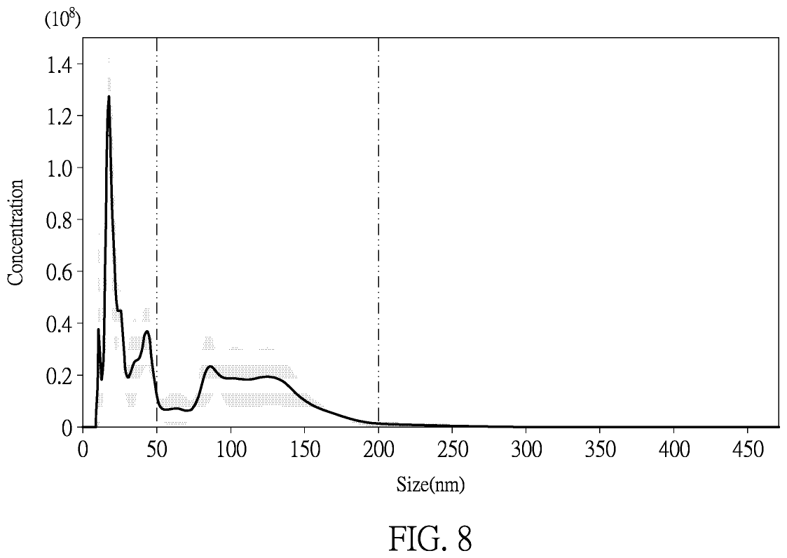

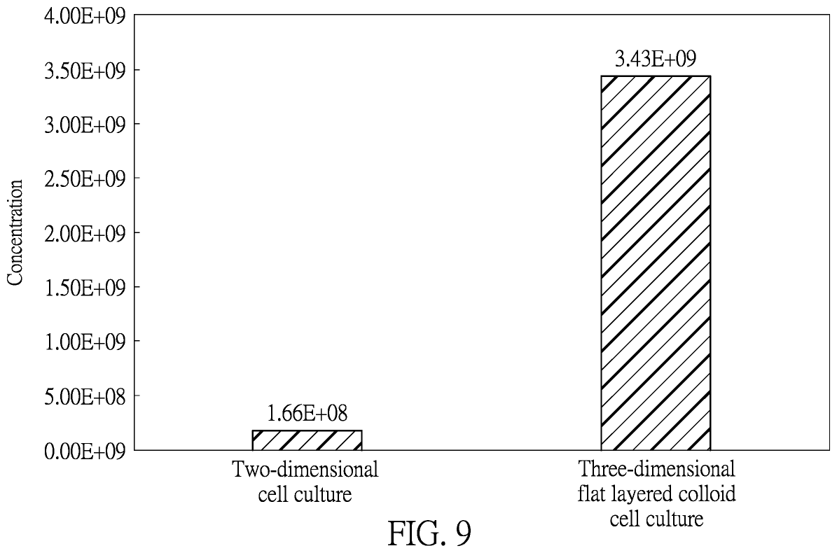

Particle size distribution and concentration of the obtained extracellular composition (i.e., exosomes) are further analyzed. As shown in FIG. 7 and FIG. 8, a major particle size distribution of the extracellular composition (i.e., exosomes) of the two-dimensional cell culture is from 50 nm to 200 nm, while a major particle size distribution of the extracellular composition (i.e., exosomes) of the three-dimensional cell culture (e.g., the three-dimensional flat layered colloid cell culture as shown in FIG. 8) is from 0 nm to 50 nm, and a concentration of the extracellular composition (i.e., exosomes) of the three-dimensional cell culture with the major particle size ranging from 0 nm to 50 nm is much higher than a concentration of the extracellular composition (i.e., exosomes) of the two-dimensional cell culture with the major particle size ranging from 50 nm to 200 nm. Further, particle concentration of the extracellular composition (i.e., exosomes) is measured by Nanoparticle Tracking Analysis (NTA). As shown in FIG. 9, a particle concentration of the extracellular composition (i.e., exosomes) of the three-dimensional cell culture (e.g., the three-dimensional flat layered colloid cell culture as shown in FIG. 9) is much higher than a particle concentration of the extracellular composition (i.e., exosomes) of the two-dimensional cell culture. Accordingly, the method for the mass production of high-concentration extracellular composition of the present disclosure is able to effectively increase the yield of the extracellular composition (i.e., exosomes) compared with the conventional method.

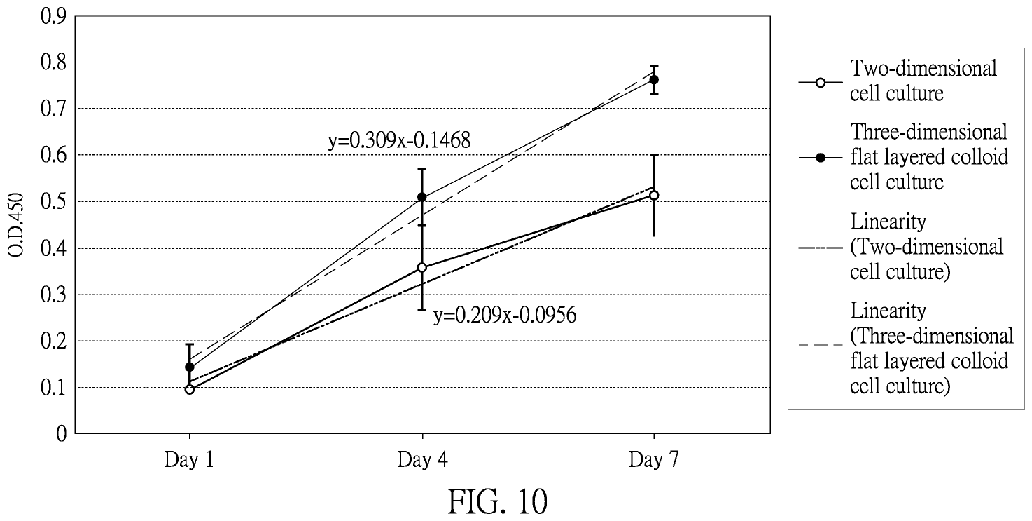

Further, efficacy of the extracellular composition (i.e., exosomes) on cell proliferation is analyzed. Effects of the extracellular compositions (i.e., exosomes) of the two-dimensional cell culture and the three-dimensional cell culture (e.g., the three-dimensional flat layered colloid cell culture as shown in FIG. 10) on the cell proliferation are analyzed by MTT assay. As shown in FIG. 10, both the extracellular compositions (i.e., exosomes) of the two-dimensional cell culture and the three-dimensional cell culture (e.g., the three-dimensional flat layered colloid cell culture as shown in FIG. 10) can enhance cell growth. However, in comparison, a doubling time of the cells treated with the extracellular composition (i.e., exosomes) of the three-dimensional cell culture is 73.01 hours, while a doubling time of the cells treated with the extracellular composition (i.e., exosomes) of the two-dimensional cell culture is 74.8 hours. In addition, a slope of a curve of cell proliferation for the cells treated with the extracellular composition (i.e., exosomes) of the three-dimensional cell culture is greater than a slope of a curve of cell proliferation for the cells treated with the extracellular composition (i.e., exosomes) of the two-dimensional cell culture, indicating that the extracellular composition (i.e., exosomes) obtained by the method for the mass production of high-concentration extracellular composition of the present disclosure has excellent efficacy for enhancing cell growth.

However, the aforementioned details are disclosed for exemplary purposes only, and are not meant to limit the scope of the present disclosure.

### Beneficial Effects of the Embodiments

In conclusion, in the method for three-dimensional cell culture using the cell growth promoting colloid and the mass production of high-concentration extracellular composition using the same provided by the present disclosure, by virtue of “performing the crosslinking treatment to the culture colloid embedding the cells, so that the culture colloid that is crosslinked has the three-dimensional structure and the cells are suspended in the culture colloid that is crosslinked,” damage to the cells can be reduced during the three-dimensional cell culture and three-dimensional growth of the cells can be supported in the culture colloid.

Further. the extracellular composition obtained using the method for three-dimensional cell culture using the cell growth promoting colloid of the present disclosure has an effective reduction in the particle size and a significant increase in the total protein concentration. Accordingly, the yield of the extracellular composition can be effectively improved, and an excellent effect of enhancing cell growth can be achieved.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for three-dimensional cell culture using a cell growth promoting colloid, comprising:
providing a culture colloid to mix with cells to embed the cells therein;
performing a crosslinking treatment to the culture colloid embedding the cells, so that the culture colloid that is crosslinked has a three-dimensional structure and the cells are suspended in the culture colloid that is crosslinked; and
performing cell culture.

2. The method according to claim 1, wherein the culture colloid includes a group consisting of sodium alginate, gelatin, gelatin methacrylamide, carboxymethyl cellulose, collagen, hyaluronic acid, gum tragacanth, gum arabic, xanthan gum, pectin, guar gum, carrageenans, and any combination thereof.

3. The method according to claim 2, wherein the culture colloid further includes a cell growth promoter.

4. The method according to claim 3, wherein the cell growth promoter includes a group consisting of extracellular matrix, collagen, fibronectin, laminin, elastin, growth factor, extracellular vesicle, and any combination thereof.

5. The method according to claim 1, wherein the cells are human-derived and animal-derived mesenchymal stem cells (MSCs), fibroblasts, epithelial cells, endothelial cells, epidermal stem cells, dental pulp/gingival stem cells, hair follicle dermal papilla cells, keratinocytes, or melanocytes.

6. The method according to claim 1, wherein the crosslinking treatment is carried out by mixing 5 vol % to 25 vol % of calcium chloride into the culture colloid mixed with the cells.

7. The method according to claim 1, wherein a storage modulus of the culture colloid is 1000 Pa to 5000 Pa.

8. A method for a mass production of high-concentration extracellular composition using the method for three-dimensional cell culture using the cell growth promoting colloid as claimed in claim 1, the method for the mass production of high-concentration extracellular composition comprising:
collecting a conditioned medium; and
performing filtration the condition medium.

9. The method according to claim 8, further comprising:
performing extraction of the extracellular composition.

10. An extracellular composition obtained using the method as claimed in claim 8.

11. An extracellular composition obtained using the method as claimed in claim 9.
